(19) United States
(12) Patent Application Publication
(10) Pub. No.: US 2025/0231196 A1
(43) Pub. Date: Jul. 17, 2025

(54) USE OF TREM-1 FOR PREDICTING AND PREVENTING POSTOPERATIVE COMPLICATIONS AFTER CARDIAC SURGERY WITH CARDIOPULMONARY BY-PASS

(71) Applicants: INSTITUT NATIONAL DE LA SANTÉ ET DE LA RECHERCHE MÉDICALE, Paris (FR); ASSISTANCE PUBLIQUE-HÔPITAUX DE PARIS (APHP), Paris (FR); CENTRE HOSPITALIER UNIVERSITAIRE DE ROUEN, Rouen (FR); SORBONNE UNIVERSITÉ, Paris (FR); UNIVERSITÉ DE ROUEN NORMANDIE, Mont-Saint-Aignan (FR); UNIVERSITÉ PARIS CITÉ, Paris (FR)

(72) Inventors: Hafid AIT-OUFELLA, Paris (FR); Thomas CLAVIER, Rouen (FR)

(21) Appl. No.: 18/698,979

(22) PCT Filed: Oct. 10, 2022
(86) PCT No.: PCT/EP2022/078117

§ 371 Date: Apr. 5, 2024

(30) Foreign Application Priority Data
Oct. 11, 2021 (EP) 21306422.3

(51) Int. Cl. G01N 33/68; A61K 38/17; C07K 16/28

(52) U.S. Cl. CPC G01N 33/6893; A61K 38/1774; C07K 16/2803; G01N 2333/70503; G01N 2800/52

(57) ABSTRACT

Cardiopulmonary by-pass (CBP) during cardiac surgery leads to deleterious systemic inflammatory response. In a prospective cohort of 46 patients older than 18 years and eligible for non-urgent cardiac surgery with CPB, measurement of sTREM-1 in the plasma was performed immediately after the onset of anesthesia (H0) and 2 and 24 hours after CBP. After CBP, sTREM-1 significantly increased at H2 and at H24 (p<0.001). Based on both baseline sTREM-1 levels and variations. 3 patterns of patients were identified. Profile 1 group with high baseline sTREM-1 levels as well as high increase, developed more severe organ failure after CBP with higher norepinephrine dose at H24, higher SOFA score and more frequently AKI at both H24 and H48. Finally, acute atrial fibrillation at H24 was more frequent in profile 1 when compared to profile 2/3, Profile 1 group had longer ICU and hospital length of stay (LOS). In conclusion, early sTREM-1 variations after cardiac surgery identified a group of patients at high risk for post-operative AKI and prolonged length of stay. Thus sTREM-1 represents a relevant biomarker and biotarget in cardiac surgery with CBP.

### FIELD OF THE INVENTION

The present invention is in the field of medicine, in particular cardiology.

### BACKGROUND OF THE INVENTION

Every year, several thousands of patients undergo cardiac surgery¹ in the world. Despite improvements in minimally invasive and endovascular methods, cardiac surgery with cardiopulmonary by-pass (CPB) remains the most frequently used procedure but is associated with life-threatening complications²⁻⁴. Postoperative complications after cardiac surgery with CPB remains too high, around 15%, as well as related mortality⁵.

Surgery procedure may account for complications but CPB by itself may also be responsible for tissue damage and organ failure due to the release of pro-inflammatory cytokines and oxidative stress by circulating leucocytes in response to both ischemia and exposure to extra-corporeal artificial surface^{6,7}. Plasma levels of cytokines after CPB correlate with postoperative complications^{8,9}. Despite improvement in the understanding of this CBP-induced “cytokine storm”, upstream engaged signaling pathways remain poorly investigated as well as anti-inflammatory targeted therapy.

TREM-1 for Triggering receptor expressed on myeloid cells 1, expressed broadly on myeloid cells is member of the immunoglobulin ‘superfamily’ and contain a single variable-type immunoglobulin domain. Engagement of TREM-1, after association with the adapter protein DAP12 (which contains an immunoreceptor tyrosine-based activation motif), triggers a signaling pathway involving ZAP70, SyK, PI3 kinase, PLC-γ, and MAP kinases¹⁰. Activation of these pathways leads to intracellular calcium mobilization, actin cytoskeleton rearrangement, and activation of transcription factors including NF-κB. In mice, engagement of TREM-1 with monoclonal agonist antibodies has been shown to promote the production of pro-inflammatory cytokines and chemokines, including IL-8, CCL2, CCL3, GM-CSF^{11,12} (18,19), as well as stimulating rapid neutrophil degranulation and oxidative burst¹³. Several studies have shown that TREM-1 participates in inflammation-induced organ damage in sepsis through cooperation with TRL-4^{14,15}. More recently, its role in acute and chronic sterile inflammation has been reported in the context of acute myocardial infarction and atherosclerosis development through the regulation of cytokine production and myeloid cell trafficking^{16,17}.

One of the features of the TREM-1 is the release of soluble receptor after stimulation. Several human studies have shown that plasma sTREM-1 could be used as diagnosis and prognosis tool for severe infections. Our group has also reported that sTREM-1 is an powerful predictive factor for 2-year mortality or MI recurrence in the context of acute myocardial infarction.

### SUMMARY OF THE INVENTION

The present invention is defined by the claim. In particular, the present invention relates to the use of sTREM-1 for predicting postoperative complications after cardiac surgery with cardiopulmonary by-pass (CPB). The present invention also relates to use of TREM-1 inhibitors for preventing postoperative complications after cardiac surgery with cardiopulmonary by-pass (CPB) in patients in need thereof.

### DETAILED DESCRIPTION OF THE INVENTION

Cardiopulmonary by-pass (CBP) during cardiac surgery leads to deleterious systemic inflammatory response. However, upstream regulating inflammatory pathways remain unknown. The inventors hypothesized that TREM-1, a myeloid receptor involved in innate immune responses is activated during CPB and shed in the plasma. In a prospective cohort study, patients older than 18 years and eligible for non-urgent cardiac surgery with CPB were included. Measurement of cytokines and sTREM-1 in the plasma were performed immediately after the onset of anesthesia (H0) and 2 and 24 hours after CBP. Patients' clinical characteristics, acute kidney injury (AKI) and length of stay (LOS) were recorded. Forty-six patients were included. After CBP, sTREM-1 significantly increased at H2 and at H24 (p<0.001). IL-6, IL-8, G-CSF and TNF-α but not IL-1β significantly increased at H2 in comparison to H0 (p<0.001) but dropped at H24. Principal component analysis (PCA) showed a close relationship between STREM-1 and IL-8. Based on both baseline sTREM-1 levels and variations, 3 patterns of patients were identified. Profile 1 group with high baseline sTREM-1 levels as well as high increase, developed more severe organ failure after CBP with higher norepinephrine dose at H24 (0.6±0.16 vs 0.1±0.03 μg/kg/min, P=0.044), higher SOFA score (3.1±3.8 vs 1±1.5, P=0.011) and more frequently AKI at both H24 (30% vs 2.8%, p=0.039) and H48 (60% vs 2.8%, p<0.001). Finally, acute atrial fibrillation at H24 was more frequent in profile 1 when compared to profile 2/3 (80% vs 19.4%, p=0.001). Profile 1 group had longer ICU and hospital length of stay (LOS) (days, 9.4 (12.6) vs 4.3 (1.5), p=0.018) and (Days 30.7 (28.5) vs 14.0 (4.5), p=0.001), when compared to profile 2/3 patients. After adjustment on age and duration of CPB, STREM-1 H2 remains associated with hospital LOS (p=0.03). sTREM-1 H2 was significantly predictive of AKI (AUC=0.86, (CI95% 0.64-1.00, p=0.008), prolonged LOS in ICU (AUC: 0.72, CI95%: 0.56-0.89, p=0.011) and in hospital (AUC: 0.72, CI95%: 0.51-0.93, p=0.016). In conclusion, early sTREM-1 variations after cardiac surgery identified a group of patients at high risk for post-operative AKI and prolonged length of stay.

### Diagnostic Methods:

Accordingly, the first object of the present invention relates to a method of predicting the postoperative outcome of a patient after cardiac surgery with cardiopulmonary by-pass (CPB) comprising determining the level of sTREM-1 in a sample obtained from the patient wherein the level of sTREM-1 indicates the postoperative outcome.

As used herein, the term “cardiac surgery” has its general meaning in the art and is meant to encompass any surgery involving the heart, including but not limited to septal defect repair, inflow/outflow tract or valve procedure, heart valve repair or replacement, surgery to place ventricular assist devices or total artificial hearts, aneurysm repair, arrhythmia treatment, and the like.

As used herein, the term “cardiopulmonary by-pass” or “CBP” has its general meaning in the art and refers to a form of extracorporeal circulation whose function is circulatory and respiratory support along with temperature management to facilitate cardia surgery. CPB circuit includes pumps, cannulae, tubing, reservoir, oxygenator, heat exchanger and arterial line filter Modern CPB machines have systems for monitoring pressures, temperature, oxygen saturation, haemoglobin, blood gases, electrolytes as well as safety features such as bubble detectors, oxygen sensor and reservoir low-level detection alarm.

As used herein, the term “postoperative outcome” refers to the likelihood that the patient has at least one postoperative complication after CBP. Typically, postoperative complications is related to tissue any damage or organ failure due to the release of pro-inflammatory cytokines and oxidative stress by circulating leucocytes in response to both ischemia and exposure to extra-corporeal artificial surface. In particular, postoperative outcome thus include but is not limited to organ failure, acute atrial fibrillation and acute kidney injury.

In particular, postoperative outcome is organ failure, acute atrial fibrillation and/or acute kidney injury.

In particular, postoperative outcome is acute atrial fibrillation and/or acute kidney injury.

As used herein, the term “organ failure” has its general meaning in the art and refers to a condition where an organ does not perform its expected function. Organ failure relates to organ dysfunction to such a degree that normal homeostasis cannot be maintained without external clinical intervention. Examples of organ failure include without limitation renal failure, liver failure, heart failure, and respiratory failure. Typically, organ failure is assessed by the Sequential Organ Failure Assessment (SOFA) score that is a simple and objective score that allows for calculation of both the number and the severity of organ dysfunction in six organ systems (respiratory, coagulatory, liver, cardiovascular, renal, and neurologic).

As used herein, the term “acute kidney injury” or “AKI” has its general meaning in the art and refers to loss of kidney function that develops within 6 days, e.g. following cardiac surgery. Kidney function may be assessed by glomerular filtration rate (GFR), i.e. the flow rate of filtered fluid through the kidney (for example, by the RIFLE class system, a GFR decrease >25% from baseline classifies risk, while injury is defined by a GFR >50% from baseline, as described in Nature Reviews Nephrology 7, 201-208; April 2011), or by creatinine clearance rate (C& or CrCl), i.e. the volume of blood plasma that is cleared of creatinine per unit time. Thus, loss of kidney function may be determined by an increase in blood levels of creatinine, e.g. a 50% or greater increase in creatinine concentrations. Typically, the AKI is assessed by the kidney disease improving global outcomes (KDIGO) score.

As used herein, the term “atrial fibrillation” has its general meaning in the art and refers to an arrhythmia in which the atrium is irregularly excited at a frequency of 450 to 600 times per minute, and that excitation wave is randomly transmitted to atrioventricular node, thus making the ventricular excitation irregular.

The method of the present invention is also particularly suitable for predicting the length of stay. As used herein, the term “length of stay” means the amount of time the patient when the patient stay at hospital (e.g. in the intensive care unit)

More particularly, the method of the present invention is also particularly suitable for predicting death of the patient.

As used herein, the term “risk” relates to the probability that an event will occur over a specific time period, as in the conversion to a postoperative complication, and can mean a subject's “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of low risk cohorts or an average population risk, which can vary by how clinical risk factors are assessed. Odds ratios, the proportion of positive events to negative events for a given test result, are also commonly used (odds are according to the formula p/(1−p) where p is the probability of event and (1−p) is the probability of no event) to no-conversion. “Risk evaluation,” or “evaluation of risk” in the context of the present invention encompasses making a prediction of the probability, odds, or likelihood that an event or disease state may occur, the rate of occurrence of the event or conversion from one disease state to another, i.e., from a normal condition to a postoperative complication or to one at risk of developing a postoperative complication. Risk evaluation can also comprise prediction of future clinical parameters, traditional laboratory risk factor values, or other indices of a postoperative complication, either in absolute or relative terms in reference to a previously measured population. The methods of the present invention may be used to make continuous or categorical measurements of the risk of conversion to a postoperative complication, thus diagnosing and defining the risk spectrum of a category of subjects defined as being at risk for a postoperative complication. In the categorical scenario, the invention can be used to discriminate between normal and other subject cohorts at higher risk for a postoperative complication.

As used herein, the term “sample” as used herein refer to a biological sample obtained for the purpose of in vitro evaluation. Typical biological samples to be used in the method according to the invention are blood samples (e.g. whole blood sample or serum sample). In some embodiments, said biological liquids comprise blood, plasma, serum, saliva and exsudates. Thus, in some embodiments, the sample is chosen from blood samples, plasma samples, saliva samples, exsudate samples and serum samples. Preferably, the sample is a blood sample, a serum sample or a plasma sample.

As used herein the term “TREM-1” has its general meaning in the art and refers to the triggering receptor expressed on myeloid cells-1 (TREM-1). TREM-1 is a member of the Ig-superfamily, the expression of which is up-regulated on phagocytic cells in the presence of bacteria or fungi (Bouchon A et al. Nature 2001; 230:1103-7). An exemplary amino acid sequence is represented by SEQ ID NO: 1. It was previously described that TREM-1 can be shed or secreted from the membrane of activated phagocytes and can be found in a soluble form in body fluids. Accordingly, the term “sTREM-1” refers to the soluble form of the human TREM-1 receptor.

>sp | Q9NP99 | TREM1 HUMAN Triggering receptor
expressed on myeloid cells 1 OS = Homo sapiens
OX = 9606 GN = TREM1 PE = 1 SV = 1
SEQ ID NO: 1
MRKTRLWGLLWMLFVSELRAATKLTEEKYELKEGQTLDVKCDYTL
EKFASSQKAWQIIRDGEMPKTLACTERPSKNSHPVQVGRIILEDY
HDHGLLRVRMVNLQVEDSGLYQCVIYQPPKEPHMLFDRIRLVVTK
GFSGTPGSNENSTQNVYKIPPTTTKALCPLYTSPRTVTQAPPKST
ADVSTPDSEINLTNVTDIIRVPVENIVILLAGGFLSKSLVFSVLF
AVTLRSFVP

The measurement of the level of sTREM-1 in the sample is typically carried out using standard protocols known in the art. For example, the method may comprise contacting the sample with a binding partner capable of selectively interacting with sTREM-1 in the sample. In some embodiments, the binding partners are antibodies, such as, for example, monoclonal antibodies or even aptamers. For example the binding may be detected through use of a competitive immunoassay, a non-competitive assay system using techniques such as western blots, a radioimmunoassay, an ELISA (enzyme linked immunosorbent assay), a “sandwich” immunoassay, an immunoprecipitation assay, a precipitin reaction, a gel diffusion precipitin reaction, an immunodiffusion assay, an agglutination assay, a complementfixation assay, an immunoradiometric assay, a fluorescent immunoassay, a protein A immunoassay, an immunoprecipitation assay, an immunohistochemical assay, a competition or sandwich ELISA, a radioimmunoassay, a Western blot assay, an immunohistological assay, an immunocytochemical assay, a dot blot assay, a fluorescence polarization assay, a scintillation proximity assay, a homogeneous time resolved fluorescence assay, a IAsys analysis, and a BIAcore analysis. The aforementioned assays generally involve the binding of the partner (ie. antibody or aptamer) to a solid support. Solid supports which can be used in the practice of the invention include substrates such as nitrocellulose (e.g., in membrane or microtiter well form); polyvinylchloride (e.g., sheets or microtiter wells); polystyrene latex (e.g., beads or microtiter plates); polyvinylidine fluoride; diazotized paper; nylon membranes; activated beads, magnetically responsive beads, and the like. An exemplary biochemical test for identifying specific proteins employs a standardized test format, such as ELISA test, although the information provided herein may apply to the development of other biochemical or diagnostic tests and is not limited to the development of an ELISA test (see, e.g., Molecular Immunology: A Textbook, edited by Atassi et al. Marcel Dekker Inc., New York and Basel 1984, for a description of ELISA tests). Therefore ELISA method can be used, wherein the wells of a microtiter plate are coated with a set of antibodies which recognize sTREM-1. A sample containing or suspected of containing sTREM-1 is then added to the coated wells. After a period of incubation sufficient to allow the formation of antibody-antigen complexes, the plate(s) can be washed to remove unbound moieties and a detectably labelled secondary binding molecule added. The secondary binding molecule is allowed to react with any captured sample marker protein, the plate washed and the presence of the secondary binding molecule detected using methods well known in the art. Measuring the level of sTREM-1 (with or without immunoassay-based methods) may also include separation of the compounds: centrifugation based on the compound's molecular weight; electrophoresis based on mass and charge; HPLC based on hydrophobicity; size exclusion chromatography based on size; and solid-phase affinity based on the compound's affinity for the particular solid-phase that is used. Once separated, said one or two biomarkers proteins may be identified based on the known “separation profile” e.g., retention time, for that compound and measured using standard techniques. Alternatively, the separated compounds may be detected and measured by, for example, a mass spectrometer. Typically, levels of immunoreactive sTREM-1 in a sample may be measured by an immunometric assay on the basis of a double-antibody “sandwich” technique, with a monoclonal antibody specific for sTREM-1 (Cayman Chemical Company, Ann Arbor, Michigan). According to said embodiment, said means for measuring sTREM-1 level are for example i) a sTREM-1 buffer, ii) a monoclonal antibody that interacts specifically with sTREM-1, iii) an enzyme-conjugated antibody specific for sTREM-1 and a predetermined reference value of sTREM-1.

In some embodiments, the level of sTREM-1 is compared to a predetermined reference value, wherein differential between the determined level of sTREM-1 and the predetermined reference value indicates the postoperative outcome.

In some embodiments, the method further comprises the steps of i) determining the level of STREM-1 in the sample obtained from the patient, ii) comparing the level of sTREM-1 with a predetermined reference value and iii) determining the postoperative outcome from said comparison. Typically, when the level of sTREM-1 is higher than the predetermined value, it is concluded that the patient is at risk of having at least one postoperative complication and conversely when the level of sTREM-1 is lower than the predetermined reference value, it is concluded that the patient is not at risk of having at least one postoperative complication.

Typically, the predetermined reference value is a threshold value or a cut-off value. A “threshold value” or “cut-off value” can be determined experimentally, empirically, or theoretically. A threshold value can also be arbitrarily selected based upon the existing experimental and/or clinical conditions, as would be recognized by a person of ordinary skilled in the art. For example, retrospective measurement of level of sTREM-1 in properly banked historical subject samples may be used in establishing the predetermined reference value. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. For example, after determining the levels of sTREM-1 in a group of reference, one can use algorithmic analysis for the statistic treatment of the measured levels of sTREM-1 in samples to be tested, and thus obtain a classification standard having significance for sample classification. The full name of ROC curve is receiver operator characteristic curve, which is also known as receiver operation characteristic curve. It is mainly used for clinical biochemical diagnostic tests. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is quite high. This algorithmic method is preferably done with a computer. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWER.SAS, CREATE-ROC.SAS, GB STAT VI0.0 (Dynamic Microsystems, Inc. Silver Spring, Md., USA), etc.

The predetermined reference value can also be relative to a number or value derived from population studies, including without limitation, subjects of the same or similar age range, subjects in the same or similar ethnic group, and subjects having the same severity of bacterial peritonitis. Such predetermined reference values can be derived from statistical analyses and/or risk prediction data of populations obtained from mathematical algorithms and computed indices. In some embodiments, the predetermined reference values are derived from the level of sTREM-1 in a control sample derived from one or more patient who do not develop a postoperative complication. Furthermore, retrospective measurement of the level of sTREM-1 in properly banked historical subject samples may be used in establishing these predetermined reference values.

In some embodiments, a cut-off value thus consists of a range of quantification values, e.g. centered on the quantification value for which the highest statistical significance value is found. For example, on a hypothetical scale of 1 to 10, if the ideal cut-off value (the value with the highest statistical significance) is 5, a suitable (exemplary) range may be from 4-6. For example, a subject may be assessed by comparing values obtained by measuring the level of sTREM-1, where values greater than 5 reveal that the patient is at risk of having at least one postoperative complication and values less than 5 reveal that the subject is not at risk of having at least one postoperative complication. In some embodiments, a subject may be assessed by comparing values obtained by measuring the level of sTREM-1 and comparing the values on a scale, where values above the range of 4-6 indicate that the subject is at risk of having at least one postoperative complication and values below the range of 4-6 indicate that the subject is not at risk of having at least one postoperative complication, with values falling within the range of 4-6 indicate that further explorations are needed to conclude whether the subject is at risk of having at least one postoperative complication.

In some embodiments, the level of sTREM-1 is determined 1, 2, 3, or 4 h after the end of CBP. In some embodiments, the level of sTREM-1 is also determined 20, 21, 23, 24, 25 hours after the end of the CBP. In some embodiments, an increase between the level determined 1, 2, 3, or 4 h after the end of CBP and the level determined 20, 21, 23, 24, 25 hours after the end of the CBP indicates the patient is at risk of having at least one postoperative complication.

Once it is concluded that the patient is at risk of having at least one postoperative complication, any therapeutic intervention may be decided. In particular, the patient is administered with a therapeutically effective amount of a TREM-1 inhibitor for preventing said postoperative complication.

Thus, the invention also refers to a method of predicting the postoperative outcome of a patient after cardiac surgery with cardiopulmonary by-pass (CPB) comprising i) determining the level of sTREM-1 in a sample obtained from the patient; ii) conclude that the patient is at risk of having at least one postoperative complication when the level of sTREM-1 is higher than a predetermined value and ii) administering a therapeutically effective amount of a TREM-1 inhibitor to the patient considered as being at risk of having at least one postoperative complication.

### Therapeutic Methods:

A further object of the present invention relates to a method of preventing a postoperative complication in a patient after cardiac surgery with cardiopulmonary by-pass (CPB) comprising administering to the patient a therapeutically effective amount of a TREM-1 inhibitor.

In some embodiments, the patient was considered as being at risk of having at least one postoperative complication by the diagnostic method of the present invention.

In other word, the invention refers to a method for preventing a postoperative complication in a patient in need thereof after cardiac surgery with cardiopulmonary by-pass (CPB) comprising i) determining the level of sTREM-1 in a sample obtained from the patient; ii) conclude that the patient is at risk of having at least one postoperative complication when the level of sTREM-1 is higher than a predetermined value and ii) administering a therapeutically effective amount of a TREM-1 inhibitor to the patient considered as being at risk of having at least one postoperative complication.

As used herein, the term “TREM-1 inhibitor” refers to any compound, chemical, antibody, or peptide, naturally occurring or synthetic, that directly or indirectly decreases the activity and/or expression of TREM-1. Functionally conservative variations of known TREM-1 inhibitors are also intended to be covered by this description. This includes, for example only, deuterated variations of known inhibitors, inhibitors comprising non-naturally occurring amino-acids, functional variations of peptide inhibitors involving a different sequence of amino acids, inhibitors created by codon variations which code for the same amino-acid sequence of a known inhibitor or functional variation thereof, versions of peptides described herein in which one or more of the amino acids can be, individually, D or L isomers. The invention also includes combinations of L-isoforms with D-isoforms.

Common TREM-1 inhibitors include peptides which may be derived from TREM-1, or TREM-like-transcript-1 (“TLT-1”). Any peptide which competitively binds TREM-1 ligands, thereby reducing TREM-1 activity and/or expression is a TREM-1 inhibitor. These peptides may be referred to as “decoy receptors.”

In some embodiments, the TREM-1 inhibitor is a peptide that is disclosed in WO2014037565. Examples of such peptides are listed below in Table A. LR17 is a known, naturally occurring direct inhibitor of TREM-1 which functions by binding and trapping TREM-1 ligand. LR12 is a 12 amino-acid peptide derived from LR17. LR12 is composed of the N-terminal 12 amino-acids from LR17. Research suggests that LR12 is an equivalent TREM-1 inhibitor when compared to LR17. LR6-1, LR6-2 and LR6-3 are all 6 amino-acids peptides derived from LR17. These peptides may function in the same manner as LR12.

TABLE A
Different peptides that can be used
as TREM-1 inhibitors

| Peptide | | |
|---|---|---|
| name | Sequence | SEQ ID |
| LR17 | LQEEDAGEYGC | SEQ ID NO: 2 |
| | MVDGAR | |
| LR12 | LQEEDAGEYGCM | SEQ ID NO: 3 |
| LR6-1 | LQEEDA | SEQ ID NO: 4 |
| LR6-2 | EDAGEY | SEQ ID NO: 5 |
| LR6-3 | GEYGCM | SEQ ID NO: 6 |
| LP17 | LQVEDSGLYQCV | SEQ ID NO: 7 |
| | IQHPP | |
| LP12 | LQVEDSGLYQCV | SEQ ID NO: 8 |
| LP6-1 | LQVEDS | SEQ ID NO: 9 |
| LP6-2 | EDSGLY | SEQ ID NO: 10 |
| LP6-3 | GLYQCV | SEQ ID NO: 11 |

In some embodiments, the TREM-1 inhibitor is a peptide derived from TLT-1 or TREM-1, in particular peptides as described herein.

In some embodiments, the TREM-1 inhibitor is a short TLT-1 peptide consisting of less than 50 amino acids, preferably consisting of between 6 and 20 amino acids, more preferably consisting of between 6 and 17 amino acids, wherein said TLT-1 peptide comprises between 6 and 20 consecutive amino acids from the human TLT-1 having an amino acid sequence as set forth in SEQ ID NO: 12 (MGLTLLLLLLLGLEGQGIVGSLPEVLQAPVGSSILVQCHYRLQDVKAQKVWCRFLPE GCQPLVSSAVDRRAPAGRRTFLTDLGGGLLQVEMVTLQEEDAGEYGCMVDGARGP QILHRVSLNILPPEEEEETHKIGSLAENAFSDPAGSANPLEPSQDEKSIPLIWGAVLLVG LLVAAVVLFAVMAKRKQGNRLGVCGRFLSSRVSGMNPSSVVHHVSDSGPAAELPLD VPHIRLDSPPSFDNTTYTSLPLDSPSGKPSLPAPSSLPPLPPKVLVCSKPVTYATVIFPGG NKGGGTSCGPAQNPPNNQTPSS); or a sequence having at least 60, 65, 70, 75, 80, 85 or 90% identity with the amino acid sequence as set forth in SEQ ID NO: 12; or a function-conservative variant or derivative thereof.

In some embodiments, the TREM-1 inhibitor is a TLT-1 peptide consisting of 6 to 12, 13, 14, 15, 16, 17, 18, 19 or 20 amino acids and comprising an amino acid sequence as set forth in SEQ ID NO: 2, SEQ ID NO: 3, SEQ ID NO: 4, SEQ ID NO: 5 or SEQ ID NO: 6, or a sequence having at least 60, 65, 70, 75, 80, 85 or 90% identity with the amino acid sequence as set forth in SEQ ID NO: 2, SEQ ID NO: 3, SEQ ID NO: 4, SEQ ID NO: 5, or SEQ ID NO: 6, respectively; or a function-conservative variant or derivative thereof.

In some embodiments, the TREM-1 inhibitor is a TLT-1 peptide comprising or consisting of an amino acid sequence as set forth in SEQ ID NO: 2, SEQ ID NO: 3, SEQ ID NO: 4, SEQ ID NO: 5, or SEQ ID NO: 6; or a sequence having at least 60, 65, 70, 75, 80, 85 or 90% identity with the amino acid sequence as set forth in SEQ ID NO: 2, SEQ ID NO: 3, SEQ ID NO: 4, SEQ ID NO: 5, or SEQ ID NO: 6, respectively; or a function-conservative variant or derivative thereof.

In some embodiments, the TREM-1 inhibitor is a TLT-1 peptide having an amino acid sequence as set forth in SEQ ID NO: 2, SEQ ID NO: 3, SEQ ID NO: 4, SEQ ID NO: 5, or SEQ ID NO: 6; or a sequence having at least 60, 65, 70, 75, 80, 85 or 90% identity with the amino acid sequence as set forth in SEQ ID NO: 2, SEQ ID NO: 3, SEQ ID NO: 4, SEQ ID NO: 5, or SEQ ID NO: 6, respectively; or a function-conservative variant or derivative thereof.

In some embodiments, the TREM-1 inhibitor is a TLT-1 peptide having an amino acid sequence as set forth in SEQ ID NO: 3, also known as LR12; or a sequence having at least 60, 65, 70, 75, 80, 85 or 90% identity with the amino acid sequence as set forth in SEQ ID NO: 3; or; or a function-conservative variant or derivative of SEQ ID NO: 3.

In some embodiments, the TREM-1 inhibitor is a short TREM-1 peptide consisting of less than 50 amino acids, preferably consisting of between 6 and 20 amino acids, more preferably consisting of between 6 and 17 amino acids, wherein said TREM-1 peptide comprises between 6 and 20 consecutive amino acids from the human TREM-1 having an amino acid sequence as set forth in SEQ ID NO: 1 or a function-conservative variant or derivative thereof.

In some embodiments, the TREM-1 inhibitor is a TREM-1 peptide consisting of 6 to 12, 13, 14, 15, 16, 17, 18, 19 or 20 amino acids and comprising an amino acid sequence as set forth in SEQ ID NO: 7, SEQ ID NO: 8, SEQ ID NO: 9, SEQ ID NO: 10 or SEQ ID NO: 11; or a sequence having at least 60, 65, 70, 75, 80, 85 or 90% identity with the amino acid sequence as set forth in SEQ ID NO: 7, SEQ ID NO: 8, SEQ ID NO: 9, SEQ ID NO: 10 or SEQ ID NO: 11, respectively; or a function-conservative variant or derivative thereof.

In some embodiments, the TREM-1 inhibitor is a TREM-1 peptide comprising or consisting of an amino acid sequence as set forth in SEQ ID NO: 7, SEQ ID NO: 8, SEQ ID NO: 9, SEQ ID NO: 10 or SEQ ID NO: 11; or a sequence having at least 60, 65, 70, 75, 80, 85 or 90% identity with the amino acid sequence as set forth in SEQ ID NO: 7, SEQ ID NO: 8, SEQ ID NO: 9, SEQ ID NO: 10 or SEQ ID NO: 11, respectively; or a function-conservative variant or derivative thereof.

In some embodiments, the TREM-1 inhibitor is a TREM-1 peptide having an amino acid sequence as set forth in SEQ ID NO: 7, SEQ ID NO: 8, SEQ ID NO: 9, SEQ ID NO: 10 or SEQ ID NO: 11 or a sequence having at least 60, 65, 70, 75, 80, 85 or 90% identity with the amino acid sequence as set forth in SEQ ID NO: 7, SEQ ID NO: 8, SEQ ID NO: 9, SEQ ID NO: 10 or SEQ ID NO: 11, respectively; or a function-conservative variant or derivative thereof.

As used herein, the term “identity” or “identical”, when used in a relationship between the sequences of two or more peptides, refers to the degree of sequence relatedness between peptides, as determined by the number of matches between strings of two or more amino acid residues. “Identity” measures the percent of identical matches between the smaller of two or more sequences with gap alignments (if any) addressed by a particular mathematical model or computer program (i.e., “algorithms”). Identity of related polypeptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math. 48, 1073 (1988). Preferred methods for determining identity are designed to give the largest match between the sequences tested. Methods of determining identity are described in publicly available computer programs. Preferred computer program methods for determining identity between two sequences include the GCG program package, including GAP (Devereux et al., Nucl. Acid. Res. \2, 387 (1984); Genetics Computer Group, University of Wisconsin, Madison, Wis.), BLASTP, BLASTN, and FASTA (Altschul et al., J. Mol. Biol. 215, 403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra). The well-known Smith Waterman algorithm may also be used to determine identity.

As used herein, the term “function-conservative variants” denotes peptides derived from the peptides as described herein, in which a given amino acid residue in a peptide has been changed without altering the overall conformation and function of said peptides, including, but not limited to, replacement of an amino acid with one having similar properties (such as, for example, similar polarity, similar hydrogen bonding potential, acidic or basic amino acid replaced by another acidic or basic amino acid, hydrophobic amino acid replaced by another hydrophobic amino acid, aromatic amino acid replaced by another aromatic amino acid). It is commonly known that amino acids other than those indicated as conserved may differ in a peptide so that the percent of amino acid sequence similarity between any two peptides of similar function may vary and may be, for example, from 70% to 99% as determined according to an alignment method such as by the Cluster Method, wherein similarity is based on the MEGALIGN algorithm. A “function-conservative variant” also includes peptides which have at least 20%, 30%, 40%, 50%, or 60% amino acid identity with the peptides as described herein, for example as determined by BLAST or FASTA algorithms, and which have the same or substantially similar properties or functions as the peptides as described herein. Preferably “function-conservative variants” include peptides which have at least 60%, 65%, 70%, 75%, 80%, 85% or 90% amino acid identity with the peptides as described herein and which have the same or substantially similar properties or functions as the peptides as described hereinabove. As used herein, the term “derivative” refers to a variation of a peptide or of a function-conservative variant thereof that is otherwise modified in order to alter the in vitro or in vivo conformation, activity, specificity, efficacy or stability of the peptide. For example, said variation may encompass modification by covalent attachment of any type of molecule to the peptide or by addition of chemical compound(s) to any of the amino-acids of the peptide. In some embodiments, the peptide or function-conservative variants or derivatives thereof as described hereinabove may have D- or L-configuration. In some embodiments, the amino acid from the amino end of the peptide or function-conservative variant or derivative thereof as described hereinabove has an acetylated terminal amino group, and the amino acid from the carboxyl end has an amidated terminal carboxy group. In addition, the peptide or function-conservative variant or derivative thereof as described hereinabove may undergo reversible chemical modifications in order to increase its bioavailability (including stability and fat solubility) and its ability to pass the blood-brain barrier and epithelial tissue. Examples of such reversible chemical modifications include esterification of the carboxy groups of glutamic and aspartic amino acids with an alcohol, thereby removing the negative charge of the amino acid and increasing its hydrophobicity. This esterification is reversible, as the ester link formed is recognized by intracellular esterases which hydrolyze it, restoring the charge to the aspartic and glutamic residues. The net effect is an accumulation of intracellular peptide, as the internalized, de-esterified peptide cannot cross the cell membrane. Another example of such reversible chemical modifications includes the addition of a further peptide sequence, which allows the increase of the membrane permeability, such as a TAT peptide or Penetratin peptide (see-Charge-Dependent Translocation of the Trojan. A Molecular View on the Interaction of the Trojan Peptide Penetratin with the 15 Polar Interface of Lipid Bilayers. Biophysical Journal, Volume 87, Issue 1, 1 Jul. 2004, Pages 332-343).

The peptides or function-conservative variants or derivatives thereof as described hereinabove may be obtained through conventional methods of solid-phase chemical peptide synthesis, following Fmoc and/or Boc-based methodology (see Pennington, M. W. and Dunn, B. N. (1994). Peptide synthesis protocols. Humana Press, Totowa.). Alternatively, the peptides or function-conservative variants or derivatives as described hereinabove may be obtained through conventional methods based on recombinant DNA technology, e.g., through a method that, in brief, includes inserting the nucleic acid sequence coding for the peptide into an appropriate plasmid or vector, transforming competent cells for said plasmid or vector, and growing said cells under conditions that allow the expression of the peptide and, if desired, isolating and (optionally) purifying the peptide through conventional means known to experts in these matters or eukaryotic cells that express the peptide. A review of the principles of recombinant DNA technology may be found, for example, in the text book entitled “Principles of Gene Manipulation: An Introduction to Genetic Engineering,” R. W. Old & S. B. Primrose, published by Blackwell Scientific Publications, 4th Edition (1989).

Additional examples of TREM-1 inhibitors include those disclosed by patent application WO 2015018936. These include, but are not limited to, antibodies directed to TREM-1 and/or STREM-1 or TREM-1 and/or sTREM-1 ligand, small molecules inhibiting the function, activity or expression of TREM-1, peptides inhibiting the function, activity or expression of TREM-1, siRNAs directed to TREM-1, shRNAs directed to TREM-1, antisense oligonucleotide directed to TREM-1, ribozymes directed to TREM-1 and aptamers which bind to and inhibit TREM-1. Antibodies have been shown to inhibit TREM-1 as well. Representative antibodies are described, for example, in U.S. Publication No. 20130309239 and U.S. Pat. No. 9,000,127. Additional examples of TREM-1 inhibitors also include those disclosed in WO2011 047097. As described in U.S. patent publications 20090081199 and 20030165875, fusion proteins between human IgG1 constant region and the extracellular domain of mouse TREM-1 or that of human TREM-1 can be used, as a decoy receptor, to inhibit TREM-1. Another TREM-1 inhibitor is TLT-1, as disclosed in Washington, et al., “A TREM family member, TLT-1, is found exclusively in the alpha-granules of megakaryocytes and platelets,” Blood. 2004 August 15; 104(4):1042-7. Additional TREM-1 inhibitors include MicroRNA 294, which has been shown to target TREM-1 by dual-luciferase assay activity. Naturally-occurring TREM-1 inhibitors include curcumin and diferuloylmethane, a yellow pigment present in turmeric. Inhibition of TREM-1 by curcumin is oxidant independent. Accordingly, curcumin and synthetic curcumin analogs, such as those described in U.S. Publication Nos. 20150087937, 20150072984, 20150011494, 20130190256; 20130156705, 20130296527, 20130224229, 20110229555; and 20030153512; U.S. Pat. Nos. 7,947,687, 8,609,723, and PCT WO 2003105751.

In some embodiments, the TREM-1 inhibitor is Nangibotide (CAS number 2014384-91-7) (Cuvier V, Lorch U, Witte S, Olivier A, Gibot S, Delor I, Garaud J J, Derive M, Salcedo-Magguilli M: A first-in-man safety and pharmacokinetics study of nangibotide, a new modulator of innate immune response through TREM-1 receptor inhibition. Br J Clin Pharmacol. 2018 October; 84 (10): 2270-2279. doi: 10.1111/bcp.13668. Epub 2018 Jul. 20).

As used herein, the term “therapeutically effective amount” refers to a sufficient amount of the TREM-1 inhibitor to prevent the postoperative complication in the patient. It will be understood, however, that the total daily usage of the agent is decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidential with the specific agent; and like factors well known in the medical arts. For example, it is well within the skill of the art to start doses of the compound at levels lower than those required to achieve the desired therapeutic effect and to gradually increase the dosage until the desired effect is achieved. However, the daily dosage of the agent may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the agent for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

Typically, the inhibitor of the present invention is combined with pharmaceutically acceptable excipients, and optionally sustained-release matrices, such as biodegradable polymers, to form pharmaceutical compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. Typically, the pharmaceutical compositions contain vehicles, which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Sterile injectable solutions are prepared by incorporating the active ingredient at the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The invention will be further illustrated by the following figures and examples. However, these examples and figures should not be interpreted in any way as limiting the scope of the present invention.

### FIGURES

FIG. 1: Kinetic of sTREM-1 and cytokines plasma levels over time. Boxplot representing the kinetics of s-TREM1, IL-1b, IL-6, IL-8, TNF-alpha and G-CSF values according to the measurement times (H0: first sampling, immediately after anesthetic induction, H2: two hours after the end of cardiopulmonary by-pass, H24: 24 hours after the end of CEC). *: adjusted p value (Holm) lower<0.05 pairwise comparison with a Wilcoxon test. **: adjusted p value (Holm) lower<0.01 pairwise comparison with a Wilcoxon test. ***: adjusted p value (Holm) lower<0.001 pairwise comparison with a Wilcoxon test. ****: adjusted p value (Holm) lower<0.001 pairwise comparison with a Wilcoxon test

FIG. 2: Length of hospital and ICU stays between patients with low and high sTREM-1 values. Estimation of Kaplan-Meir survival curves, representing the ICU and hospitalization length of stay (LOS) (days) between patients with low or high sTREM-1 values at H2. The curves are compared by a log-rank test. A high level of sTREM-1 was defined as a level higher than the third quantile of either the cohort (367 pg/ml for H2 and 386 μg/ml for H24). A: ICU LOS between patients with high or low levels of sTREM-1 at H2. B: ICU LOS between patients with high or low levels of sTREM-1 to H4. C: Hospitalization LOS between patients with high or low levels of sTREM-1 to H2. D: Hospitalization LOS between patients with high or low levels of sTREM-1 at H24.

### EXAMPLE

### Methods

### Study Scheme

A prospective observational study was conducted between June 2018 and April 2019 in the intensive Care Unit of cardiac Surgery in a tertiary teaching hospital¹⁸. Patients older than 18 years of age, eligible for cardiac surgery with CPB of more than 1 hour were included. Patients under guardianship, pregnant women, patients undergoing an emergency surgical procedure, and patients with a planned CEC duration of less than 1 hour were excluded.

Anesthesic induction was achieved by the combination of hypnotic (propofol or etomidate) and morphinic (Sufentanil or Remifentanil) drugs. Maintenance of anaesthesia was done with propofol. A bolus of heparin was administered intravenously before the start of CPB and antagonized by protamine sulfate at the end of the procedure. Cardioplegia was performed with either hyperkalemic solution enriched with beta-blocker or by Custodiol. Mean arterial blood pressure was maintained between 50 and 70 mmHg. Other therapies were left to the choice of the clinician in charge of the patient.

### Data Collection.

For each patient, sex, age and body mass index (BMI), duration of CPB, type and duration of surgery were collected. Requirement of vasopressor and invasive mechanical ventilation were evaluate 2 and 24 hours (H2 and H24) after the end of CPB. Acute kidney injury was assessed by the kidney disease improving global outcomes score (KDIGO) classification¹⁹ at H24.

Cytokine and sTREM-1 Dosage

Biological blood samples were all collected from the arterial cannula. Blood samples were taken just after anesthetic induction (H0) and then 2 and 24 hours (H2 and H24) after the end of CPB. Blood samples were stored in EDTA tubes (4 ml) and then immediately centrifuged at 3000 G for 15 minutes. The plasma was then collected in microtubes and frozen at −80° C. until analysis. Plasma concentrations of soluble TREM-1 (pg/mL) were determined in duplicate by enzyme linked immunosorbent assay (RnD Systems®) and the mean value was recorded. The levels of 5 cytokines/growth factor (IL-1β, IL-6, IL-8,TNF-α, G-CSF) were measured by Luminex technology according to the manufacturer's instructions (Bio-Plex, Bio-Rad, 5-Plex Assays panel, Marnes-la-Coquette, France).

### Statistical Analysis

Data are expressed as mean (standard deviation), medians or proportion (%). The kinetics of cytokines and sTREM-1 were assessed at different times points by repeated measures ANOVA or a Friedman test. The comparison of mean or median values was performed by applying a pairwise comparison test of Student, Welch or Wilcoxon depending on the distribution of variables and equality of variances. For categorical variables, Fisher's exact test or the Chi-square test were used. To study the relationship between sTREM-1 and the categorical variables a multiple linear regression was used. To study the similarity of the patients according to the level of sTREM-1, a hierarchical classification was carried out using the package ‘pheatmap’²⁰. The relationship between cytokines was evaluated by applying a Pearson correlation matrix using the ‘rstatix’ package²¹. Principal component analysis (PCA) and multidimensional scaling (MDS) were applied on the basis of the results of the matrix correlation using the ‘FactorMineR’ package^{22,23}. The relationship of sTREM-1 with other cytokines was performed by applying a focused principal component analysis according to the method of Falissard et al.²⁴ using the ‘Psy’ package²⁴. The predictive capacity of s-TREM1 was evaluated by a ROC curve using ‘pROC’ and ‘verification’ packages^{25,26} Best cut-off for sensitivity and specificity was calculated by Youden index. Kaplan-Meier estimation was used to assess the relationship between sTREM-1 and ICU/hospitalization LOS using the package ‘survival’²⁷. The comparison of survival curve was done by log-rank test. Survival curves adjustment was done by Cox proportional-hazards model. Because ICU monitoring is standardized, the threshold of 5 days was used for a prolonged ICU stay definition. For the hospitalization LOS, a threshold corresponding to a duration greater than the third quantile was retained (18 days). A sTREM-1 level higher than the third quantile was defined as high. Acute renal failure was defined according to the KDIGO classification as a score higher than 1¹⁹.

The significance level of 5% of the p-value was retained. Statistical adjustments of the p-value were done by the Holm method²⁸. All statistical tests were performed using the free software R version 4.0.3²⁹.

### Ethics and Consent

The study (N°2017/179/HP) was approved by the South Mediterranean II Ethics Committee (n° CPP 2017-A03375-48) in accordance with French legislation and the ethical principles of the Declaration of Helsinki. All patients included in the present study expressed their written consent.

### Results

### Patients' Characteristics

Forty-six patients were included and their main characteristics are shown in Table 1. The median age was 68±11 years, mainly male (65%) and SAPS II was 34±10. Heart surgery was done for valve disease (56.5%), coronary artery diseases (19.6%) or both (23.9%). Mean duration of CBP was 121±40 minutes. At H24, 13 patients (28.3%) required support organ therapy including vasopressor infusion (n=9, 19.6%) and mechanical ventilation (n=4, 8.7%) and 4 patients developed acute kidney failure (KDIGO>1, 8.7%).

No death was recorded. The median length of stay was 5.3±6.1 days in ICU and 17.6±15.0 days in hospital.

Kinetic of Plasma Cytokines and sTREM-1

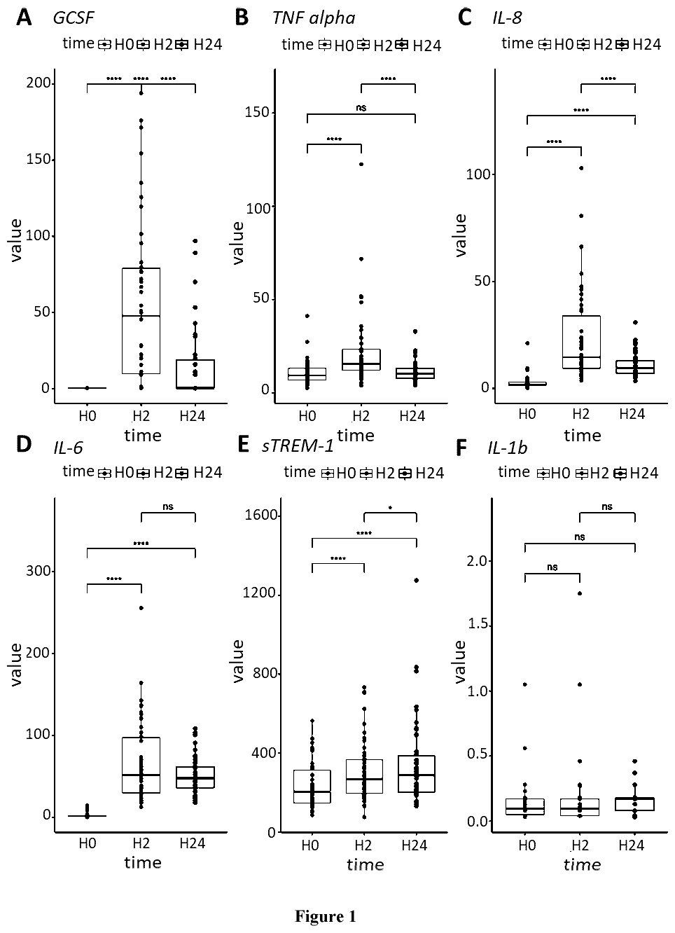

During CBP, we observed significant variations of IL-6, IL-8, G-CSF, TNF-α and sTREM-1 plasma levels (Friedman test, p.adjust<0.001 for all biomarkers) but no change of IL-1β (p=0.671) (FIG. 1). Between H0 and H2, sTREM-1 and all cytokines except IL-1β significantly increased (paired test, p.adjust<0.001). Between H2 and H24, sTREM-1 levels increased much more (p.adjust=0.019) whereas cytokine levels either decreased (ie. IL-8, G-CSF, TNF-α) or remained stable (ie. IL-6) (FIG. 1). H2 and H24 sTREM-1 levels significantly correlate with CBP duration (data not shown).

H2 sTREM-1 correlates with cytokines, except IL-1β, the correlation being more important for IL-8 (H24, r=0.62 (95% CI=0.18-0.85), p.adjust=<0.0001). H24 sTREM-1 significantly correlated with H24 IL-8 (r=0.67 (95% CI=0.26-0.87), p.adjust=<0.0001). Multivariate analysis by FPCA confirmed the close relationship between sTREM-1 and IL-8 (data not shown). MDS applied to the matrix correlation showed two aggregated groups of biomarkers, one group including TNF-α, IL-8, IL-6 and G-CSF at H2 and another group including IL-8 and STREM-1 (data not shown).

Patient Profiling Using sTREM-1 Kinetic

Baseline sTREM-1 levels was different from one patient to the other as well as kinetic after CBP. The hierarchical clustering allowed to identify three different patient patterns (data not shown): patients with high baseline levels of sTREM-1 and high increase between H2 and H24 (Profile 1), patients with moderate sTREM-1 levels which remained stable (Profile 2) or decreased over time (Profile 3).

Clinical parameters according to sTREM-1 profile are depicted in Table 2. Duration of both CBP and surgery was not different between groups. However, when compared to profile 2/3, profile 1 patients developed more severe organ failure after CBP with higher norepinephrine dose at H24 (0.6±0.16 vs 0.1±0.03 μg/kg/min, P=0.044), higher SOFA score (3.1±3.8 vs 1±1.5, P=0.011) and more frequently AKI at both H24 (30% vs 2.8%, p=0.039) and H48 (60% vs 2.8%, p<0.001). Finally, acute atrial fibrillation at H24 was more frequent in profile 1 when compared to profile 2/3 (80% vs 19.4%, p=0.001). No significant difference was observed between profile 2 and profile 3 groups. Compared to profile 2/3, profile 1 patients had longer length of stay in both hospital and ICU (respectively, log rank p value=0.024 and 0.025). After adjustment on age and CPB duration, the relationship between H2 sTREM-1 and hospital length of stay (Cox regression, p=0.03) and between H24 sTREM-1 and both ICU and hospital length of stay (Cox regression, p=0.029 and 0.011, respectively) remained significant.

Predictive Value of sTREM-1

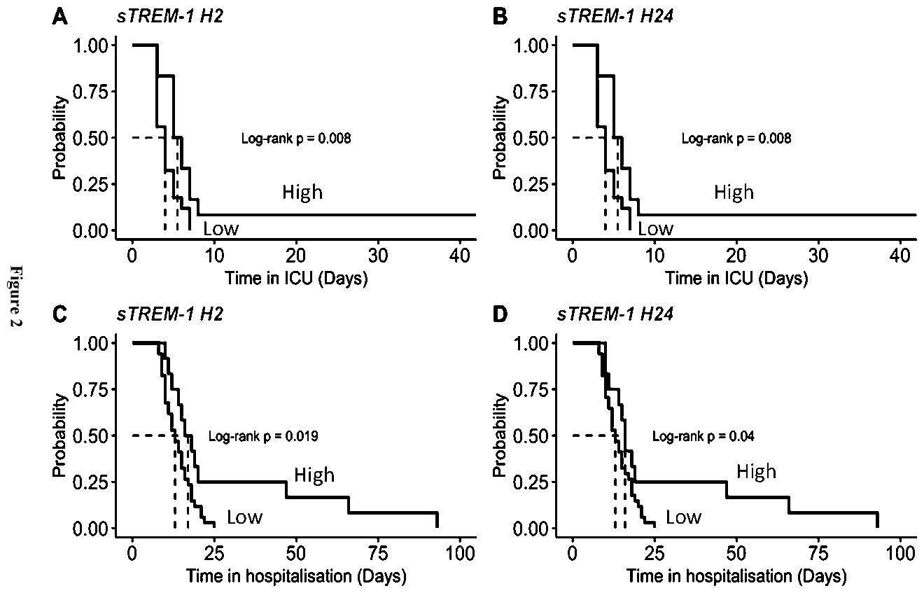

The area under the curve of sTREM-1 at H2 to predict AKI was 0.86 (CI95%: 0.64-1, Se=0.75, Sp=0.97, cut-off=586 μg/ml, p=0.008). The ability of sTREM-1 values at H2 to predict prolonged length of stay in ICU (>5 days) was 0.72 (CI95%: 0.56; 0.89, Se=0.83, Sp=0.50, cut-off=243 pg/ml, p=0.011) and to predict prolonged length of stay in hospital (>18 days) was 0.72 (CI95%: 0.51-0.93, Se=0.80, Sp=0.61, cut-off-268 pg/ml, p=0.016) (FIG. 2).

### Discussion

In this prospective cohort study in the context of non-urgent cardiac surgery, we observed an early and sustained increase of sTREM-1 after CBP whereas inflammatory cytokine levels increased at H2 but decreased later on. By performing a hierarchical clustering based on STREM-1 kinetic, we identified a group of patients who developed more frequently AKI and had prolonged length of stay in both ICU and hospital.

Our results regarding cytokine kinetics are consistent with previous studies showing an increase of TNF-α, IL-6, and IL-8 within minutes after the start of surgery^{6,30}, changes being correlated with CBP and ischemia time. sTREM-1 levels correlated with pro-inflammatory cytokine levels supporting gain- and loss-of-function experimental studies which showed that TREM-1 engagement drive cytokine production through NF-κB activation^{31,32}. Here, mediators that stimulate TREM-1 remain unknown but several candidates could be proposed. First, circulating endotoxin, detected during CBP, could stimulate TLR-4 which in turn may promote both TREM-1 expression and activation³³. Angiotensin II released during CBP could also activate TREM-1 through ATIR receptor³⁴. In this study, we did not analyze directly TREM-1 expression on circulating immune cells but we speculated that neutrophils were the main cellular source of sTREM-1. Such an hypothesis is supported by 3 elements 1/TREM-1 is expressed by almost all circulating neutrophils³⁵ 2/IL-8, which highly correlates with sTREM-1, is mainly produced by neutrophils³⁶ 3/Neutrophils are known to be activated by extra-corporeal artificial surface³⁷⁻³⁹. However, we cannot exclude sTREM-1 release from circulating non-classical monocytes⁴⁰ or endothelial cells³³. We did not observe any significant variation of IL-1β during CBP, which is also consistent with previous studies. This result is not clearly understood but could be due to intraoperative hypothermia, which affect intracellular metabolism of IL-1B⁴².

Using sTREM-1 kinetic, we identified a group of patients at high risk for AKI. The association between sTREM-1 levels and AKI has been reported in septic shock context but the pathophysiological link between this receptor activation and kidney dysfunction remains unknown. We speculated that TREM-1 may promote kidney damage through the stimulation of proinflammatory cytokine production as well as oxidative stress^{43,44} or through chemokine production which in turn orchestrates the recruitment of pathogenic immune cells in the kidney^{45,46} Finally, TREM-1 expressed by renal epithelial cells may promote kidney damage through apoptosis and autophagy induction⁴⁷.

In our study, we showed that high sTREM-1 levels were associated with prolonged ICU/hospital length of stay. This association remains significant after adjustment for age and duration of CPB, ruling out several potential confounders. However, we did not have any clear explanation for this association. TREM-1 engagement may be responsible for enhanced systemic inflammation which negatively impact on cardiac function, vascular tone and infection susceptibility and in fine slow down post-operative recovery. Unfortunately, in this study, we did not record secondary infections or heart failure after surgery.

Our results prompt us to consider that TREM-1, a master regulator of cytokine/chemokine production is involved in the deleterious inflammatory response following CBP. TREM-1 inhibition represents an interesting strategy to be tested in this context to limit post-operative complications such as AKI and to shorten hospital length of stay. Our group and others have developed a pharmacological TREM-1 blocker, named LR-12, which provided benefits in experimental chronic diseases such and atherosclerosis¹⁷ and aortic aneurysm³⁴ and in acute injury including sepsis and acute myocardial infarction¹⁶. A phase IIa trial in septic shock patients has been recently conducted showing that TREM-1 blockade may be safe in critically ill patients⁴². This targeted immunomodulatory strategy is therefore credible in cardiac surgery patients.

### Conclusion

In the context of cardiac surgery, TREM-1 may be involved in CBP-related inflammatory response and post-operative complications both being responsible for prolonged length of stay. It would therefore very interesting to administer the patients at risk of said postoperative complication with TREM-1 inhibitors.

### Tables

TABLE 1
characteristics of included patients

| Demographic characteristics | | |
|---|---|---|
| Number of patients (n) | 46 | |
| Age [years, mean (SD)] | 68 | (11) |
| Gender (F/M) | 16/36 | |
| BMI [kg/m², mean (SD)] | 28.2 | (4.7) |
| Per-operative characteristics | | |
| Type of surgery [n (%)]: | | |
| CABG | 9 | (19.6) |
| Valve surgery | 26 | (56.5) |
| CABG + valve surgery | 11 | (23.9) |
| Duration of surgery [min, mean (SD)] | 223 | (54) |
| Duration of CPB [min, mean (SD)] | 121 | (40) |
| Post-operative characteristics | | |
| SAPS II [score, mean (SD)] | 34 | (10) |
| Vasopressor H24 [n (%)] | 9 | (19.6) |
| Mechanical ventilation H24 [n (%)] | 4 | (8.7) |
| Acute kidney injury [KDIGO > 1, [n (%)] | 4 | (8.7) |
| Length of ICU stay [days, mean (SD)] | 5.3 | (6.1) |
| Length of hospital stay [days, mean(SD)] | 17.6 | (15.0) |
| Death [n (%)] | 0 | (0) |

This table represents the pre, per and post-operative characteristics of the patients. The values are expressed as mean ± standard deviation (SD), number (n) and percentage (%). F: female. M: male. CABG: coronary artery bypass graft. Min: minutes. CPB: extracorporeal circulation. ICU: intensive care unit. KDIGO: Kidney Disease International Outcomes score¹⁹. SAPS II = simplified acute physiology score⁴⁶

TABLE 2
Comparison of clinical parameters between
profile 1 patients and profile 2/3 patients

| | | | Profile | | |
|---|---|---|---|---|---|
| | Profile 1 | | 2 or 3 | | p value |
| Numbers of patients (n) | 10 | | 36 | | |
| Age [years, mean (SD)] | 76.04 | (5.1) | 66.06 | (11.2) | 0.009 |
| Gender [male, n (%)] | 6 | (60.0) | 30 | (83.3) | 0.250 |
| BMI [kg · m², | 30.13 | (3.8) | 27.63 | (4.9) | 0.144 |
| mean (SD)] | | | | | |
| Blood cardioplegia | 3 | (33.3) | 16 | (44.4) | 0.821 |
| [n (%)] | | | | | |
| Duration of CPB | 133.90 | (52.2) | 118.00 | (36.0) | 0.271 |
| [min, mean (SD)] | | | | | |
| Duration of surgery | 224.90 | (66.3) | 222.83 | (50.8) | 0.916 |
| [min, mean (SD)] | | | | | |
| Norepinephrine H24 | 0.6 | (0.16) | 0.1 | (0.03) | 0.044 |
| [μg/kg/min, mean (SD)] | | | | | |
| Assisted ventilation H24 | 2 | (20.0) | 2 | (5.6) | 0.424 |
| [n (%)] | | | | | |
| AKI H24 [n(%)] | 3 | (30.0) | 1 | (2.8) | 0.039 |
| AKI H48 [n(%)] | 6 | (60) | 1 | (2.8) | <0.001 |
| AF H24 [n(%)] | 8 | (80) | 7 | (19.4) | 0.001 |
| SOFA H48 [mean (SD)] | 3.1 | (3.8) | 1 | (1.5) | 0.011 |
| Duration of ICU stay | 9.40 | (12.5) | 4.25 | (1.5) | 0.018 |
| [days, mean (SD)] | | | | | |
| Duration of hospital stay | 30.70 | (28.4) | 14.00 | (4.5) | 0.001 |
| [days, mean (SD)] | | | | | |

Comparison of clinical parameters between profile 1 (increase of sTREM-1) and profiles 2/3 patients (relative stabilization or decrease of sTREM-1). Values expressed as mean ± standard deviation (SD) and as number (n) and percentage (%). F: female. M: male. CABG: coronary artery bypass graft. Min: minutes. CPB: extracorporeal circulation. IGS2: prognostic score [reference]. KDIGO: Kidney Disease International Outcomes score¹⁹.

### REFERENCES

Throughout this application, various references describe the state of the art to which this invention pertains. The disclosures of these references are hereby incorporated by reference into the present disclosure. 10
O'Brien, S. M. et al. The Society of Thoracic Surgeons 2018 Adult Cardiac Surgery Risk Models: Part 2-Statistical Methods and Results. Ann. Thorac. Surg. 105, 1419-1428 (2018).
2. Mejia, O. A. V. et al. Analysis of &gt; 100,000 Cardiovascular Surgeries Performed at the Heart Institute and a New Era of Outcomes. Arq. Bras. Cardiol. 114, 603-612 (2020).
3. Doenst, T. et al. Cardiac Surgery 2019 Reviewed. Thorac. Cardiovasc. Surg. 68, 363-376 (2020).
4. Makkar, R. R. et al. Five-Year Outcomes of Transcatheter or Surgical Aortic-Valve Replacement. N. Engl. J. Med. 382, 799-809 (2020).
5. Crawford, T. C. et al. Complications After Cardiac Operations: All Are Not Created 20 Equal. Ann. Thorac. Surg. 103, 32-40 (2017).
6. Warltier, D. C., Laffey, J. G., Boylan, J. F. & Cheng, D. C. H. The Systemic Inflammatory Response to Cardiac Surgery: Implications for the Anesthesiologist. Anesthesiology 97, 215-252 (2002).
7. Squiccimarro, E. et al. Prevalence and Clinical Impact of Systemic Inflammatory Reaction After Cardiac Surgery. J. Cardiothorac. Vasc. Anesth. 33, 1682-1690 (2019).
8. Paparella, D., Yau, T. M. & Young, E. Cardiopulmonary bypass induced inflammation: pathophysiology and treatment. An update. Eur. J. Cardiothorac. Surg. 21, 232-244 (2002).
9. Turer, A. T. & Hill, J. A. Pathogenesis of Myocardial Ischemia-Reperfusion Injury and Rationale for Therapy. Am. J. Cardiol. 106, 360-368 (2010).
10. Tammaro, A. et al. TREM-1 and its potential ligands in non-infectious diseases: from biology to clinical perspectives. Pharmacol. Ther. 177, 81-95 (2017).
11. Dower, K., Ellis, D. K., Saraf, K., Jelinsky, S. A. & Lin, L.-L. Innate immune responses to TREM-1 activation: overlap, divergence, and positive and negative cross-talk with bacterial lipopolysaccharide. J. Immunol. Baltim. Md 1950 180, 3520-3534 (2008).
12. Bleharski, J. R. et al. A role for triggering receptor expressed on myeloid cells-1 in host defense during the early-induced and adaptive phases of the immune response. J. Immunol. Baltim. Md 1950 170, 3812-3818 (2003).
13. Radsak, M. P., Salih, H. R., Rammensee, H.-G. & Schild, H. Triggering receptor expressed on myeloid cells-1 in neutrophil inflammatory responses: differential regulation of activation and survival. J. Immunol. Baltim. Md 1950 172, 4956-4963 (2004).
14. Arts, R. J. W. et al. TREM-1 interaction with the LPS/TLR4 receptor complex. Eur. Cytokine Netw. 22, 11-14 (2011).
15. Haselmayer, P. et al. Signaling Pathways of the TREM-1- and TLR4-Mediated Neutrophil Oxidative Burst. J. Innate Immun. 1, 582-591 (2009).
16. Boufenzer, A. et al. TREM-1 Mediates Inflammatory Injury and Cardiac Remodeling Following Myocardial Infarction. Circ. Res. 116, 1772-1782 (2015).
17. Joffre, J. et al. Genetic and Pharmacological Inhibition of TREM-1 Limits the Development of Experimental Atherosclerosis. J. Am. Coll. Cardiol. 68, 2776-2793 (2016).
18. Clavier, T. et al. A Weak Response to Endoplasmic Reticulum Stress Is Associated With Postoperative Organ Failure in Patients Undergoing Cardiac Surgery With Cardiopulmonary Bypass. Front. Med. 7, 613518 (2020).
19. Summary of Recommendation Statements. Kidney Int. Suppl. 2, 8-12 (2012).
20. CRAN Package pheatmap. https://cran.r-project.org/web/packages/pheatmap/index.html.
21. Kassambara, A. Pipe-Friendly Framework for Basic Statistical Tests [R package rstatix version 0.7.0]. https://CRAN.R-project.org/package=rstatix (2021).
22. Lê, S., Josse, J. & Husson, F. FactoMineR: An R Package for Multivariate Analysis. J. Stat. Softw. 25, 1-18 (2008).
23. FactoMineR: Multivariate Exploratory Data Analysis and Data Mining. https://CRAN.R-project.org/package=FactoMineR.
24. psy: Various procedures used in psychometry. https://CRAN.R-project.org/package=psy.
25. Robin, X. et al. pROC: Display and Analyze ROC Curves. (2021).
26. verification: Weather Forecast Verification Utilities. https://CRAN.R-project.org/package=verification.
27. Therneau, T. M. Survival Analysis [R package survival version 3.2-11]. https://CRAN.R-project.org/package=survival (2021).
28. Holm, S. A Simple Sequentially Rejective Multiple Test Procedure. Scand. J. Stat. 6, 65-70 (1979).
29. Download R-4.1.0 for Windows. The R-project for statistical computing. https://cran.r-project.org/bin/windows/base/.
30. Wan, S., LeClerc, J. L. & Vincent, J. L. Cytokine responses to cardiopulmonary bypass: lessons learned from cardiac transplantation. Ann. Thorac. Surg. 63, 269-276 (1997).
31. Liu, F. et al. TREM1: A positive regulator for inflammatory response via NF-κB pathway in A549 cells infected with Mycoplasma pneumoniae. Biomed. Pharmacother. Biomedecine Pharmacother. 107, 1466-1472 (2018).
32. Fortin, C. F., Lesur, O. & Fulop, T. Effects of TREM-1 activation in human neutrophils: activation of signaling pathways, recruitment into lipid rafts and association with TLR4. Int. Immunol. 19, 41-50 (2007).
33. Arts, R. J. W. et al. TREM-1 interaction with the LPS/TLR4 receptor complex. Eur. Cytokine Netw. 22, 11-14 (2011).
34. Vandestienne, M. et al. TREM-1 orchestrates angiotensin II-induced monocyte trafficking and promotes experimental abdominal aortic aneurysm. J. Clin. Invest. 131, (2021).
35. Bouchon, A., Dietrich, J. & Colonna, M. Cutting Edge: Inflammatory Responses Can Be Triggered by TREM-1, a Novel Receptor Expressed on Neutrophils and Monocytes. J. Immunol. 164, 4991-4995 (2000).
36. Altstaedt, J., Kirchner, H. & Rink, L. Cytokine production of neutrophils is limited to interleukin-8. Immunology 89, 563-568 (1996).
37. Larson, D. F., Bowers, M. & Schechner, H. W. Neutrophil activation during cardiopulmonary bypass in paediatric and adult patients. Perfusion 11, 21-27 (1996).
38. Beaubien-Souligny, W., Neagoe, P.-E., Gagnon, D., Denault, A. Y. & Sirois, M. G. Increased Circulating Levels of Neutrophil Extracellular Traps During Cardiopulmonary Bypass. CJC Open 2, 39-48 (2020).
39. Kawahito, K. et al. Enhanced responsiveness of circulatory neutrophils after cardiopulmonary bypass: increased aggregability and superoxide producing capacity. Artif. Organs 24, 37-42 (2000).
40. Klesney-Tait, J., Turnbull, I. R. & Colonna, M. The TREM receptor family and signal integration. Nat. Immunol. 7, 1266-1273 (2006).
41. Jolly, L. et al. Targeted endothelial gene deletion of triggering receptor expressed on myeloid cells-1 protects mice during septic shock. Cardiovasc. Res. 114, 907-918 (2018).
42. Haeffner-Cavaillon, N. et al. Induction of interleukin-1 production in patients undergoing cardiopulmonary bypass. J. Thorac. Cardiovasc. Surg. 98, 1100-1106 (1989).
43. Bouchon, A., Facchetti, F., Weigand, M. A. & Colonna, M. TREM-1 amplifies inflammation and is a crucial mediator of septic shock. Nature 410, 1103-1107 (2001).
44. Wang, Y. & Bellomo, R. Cardiac surgery-associated acute kidney injury: risk factors, pathophysiology and treatment. Nat. Rev. Nephrol. 13, 697-711 (2017).
45. Tanaka, S. et al. Vascular adhesion protein-1 enhances neutrophil infiltration by generation of hydrogen peroxide in renal ischemia/reperfusion injury. Kidney Int. 92, 154-164 (2017).
46. Hammond, M. E. et al. IL-8 induces neutrophil chemotaxis predominantly via type I IL-8 receptors. J. Immunol. Baltim. Md 1950 155, 1428-1433 (1995).
47. Pan, P. et al. TREM-1 promoted apoptosis and inhibited autophagy in LPS-treated HK-2 cells through the NF-κB pathway. Int. J. Med. Sci. 18, 8-17 (2021).
48. François, B. et al. Nangibotide in patients with septic shock: a Phase 2a randomized controlled clinical trial. Intensive Care Med. 46, 1425-1437 (2020).

1. A method of predicting the postoperative outcome of a patient after cardiac surgery with cardiopulmonary by-pass (CPB) and treating the patient for a post-operative complication, comprising
determining a level of sTREM-1 in a sample obtained from the patient, and
administering a therapeutically effective amount of a TREM-1 inhibitor to a patient identified as having a sTREM-1 level that is higher than a predetermined reference value.

2. The method of claim 1 for wherein the post-operative complication is organ failure, acute atrial fibrillation or acute kidney injury.

3. The method of claim 1 wherein the method predicts a length of stay of the patient.

4. The method of claim 1 wherein the sample is a plasma sample.

5. The method of claim 1 wherein the level of sTREM-1 is compared to the predetermined reference value and a difference between the level of sTREM-1 and the predetermined reference value indicates the postoperative outcome.

6. The method of claim 1 further comprising the steps of i) comparing the level of sTREM-1 with the predetermined reference value and ii) determining the postoperative outcome from said comparison.

7. The method of claim 1 wherein an increase between a level of STREM-1 determined 1, 2, 3, or 4 h after the end of CBP and a level of sTREM-1 determined 20, 21, 23, 24, or 25 hours after the end of the CBP indicates that the patient is at risk of having at least one postoperative complication.

8. A method of preventing a postoperative complication after cardiac surgery with cardiopulmonary by-pass (CPB) comprising administering to the patient a therapeutically effective amount of a TREM-1 inhibitor.

9. The method of claim 8 wherein the TREM-1 inhibitor is an antibody directed to TREM-1.

10. The method of claim 8 wherein the TREM-1 inhibitor is a peptide selected from the group consisting of SEQ ID NO:2, SEQ ID NO:3; SEQ ID NO:4, SEQ ID NO: 5, SEQ ID NO:6, SEQ ID NO:7, SEQ ID NO:8, SEQ ID NO:9, SEQ ID NO:10 and SEQ ID NO:11.

11. The method of claim 8 wherein the TREM-1 inhibitor is a TLT-1 peptide consisting of less than 50 amino acids, wherein said TLT-1 peptide comprises between 6 and 20 consecutive amino acids from the human TLT-1 having an amino acid sequence as set forth in SEQ ID NO: 12, or a sequence having at least 60, 65, 70, 75, 80, 85 or 90% identity with the amino acid sequence as set forth in SEQ ID NO: 12; or a function-conservative variant or derivative thereof.

12. The method of claim 8 wherein the TREM-1 inhibitor is a TLT-1 peptide consisting of 6 to 12, 13, 14, 15, 16, 17, 18, 19 or 20 amino acids and comprising an amino acid sequence as set forth in SEQ ID NO: 2, SEQ ID NO: 3, SEQ ID NO: 4, SEQ ID NO: 5 or SEQ ID NO: 6; or a sequence having at least 60, 65, 70, 75, 80, 85 or 90% identity with the amino acid sequence as set forth in SEQ ID NO: 2, SEQ ID NO: 3, SEQ ID NO: 4, SEQ ID NO: 5, or SEQ ID NO: 6, respectively; or a function-conservative variant or derivative thereof.

13. The method of claim 8 wherein the TREM-1 inhibitor is a TLT-1 peptide comprising or consisting of an amino acid sequence as set forth in SEQ ID NO: 2, SEQ ID NO: 3, SEQ ID NO: 4, SEQ ID NO: 5, or SEQ ID NO: 6; or a sequence having at least 60, 65, 70, 75, 80, 85 or 90% identity with the amino acid sequence as set forth in SEQ ID NO: 2, SEQ ID NO: 3, SEQ ID NO: 4, SEQ ID NO: 5, or SEQ ID NO: 6, respectively; or a function-conservative variant or derivative thereof.

14. The method of claim 8 wherein the TREM-1 inhibitor is a TLT-1 peptide having an amino acid sequence as set forth in SEQ ID NO: 2, SEQ ID NO: 3, SEQ ID NO: 4, SEQ ID NO: 5, or SEQ ID NO: 6; or a sequence having at least 60, 65, 70, 75, 80, 85 or 90% identity with the amino acid sequence as set forth in SEQ ID NO: 2, SEQ ID NO: 3, SEQ ID NO: 4, SEQ ID NO: 5, or SEQ ID NO: 6, respectively; or a function-conservative variant or derivative thereof.

15. The method of claim 8 wherein the TREM-1 inhibitor is a TLT-1 peptide having an amino acid sequence as set forth in SEQ ID NO: 3; or a sequence having at least 60, 65, 70, 75, 80, 85 or 90% identity with the amino acid sequence as set forth in SEQ ID NO: 3; or a function-conservative variant or derivative of SEQ ID NO: 3.

16. The method of claim 8 wherein the TREM-1 inhibitor is a short TREM-1 peptide consisting of less than 50 amino acids, wherein said TREM-1 peptide comprises between 6 and 20 consecutive amino acids from the human TREM-1 having an amino acid sequence as set forth in SEQ ID NO: 1 or a function-conservative variant or derivative thereof.

17. The method of claim 8 wherein the TREM-1 inhibitor is a TREM-1 peptide consisting of 6 to 12, 13, 14, 15, 16, 17, 18, 19 or 20 amino acids and comprising an amino acid sequence as set forth in SEQ ID NO: 7, SEQ ID NO: 8, SEQ ID NO: 9, SEQ ID NO: 10 or SEQ ID NO: 11; or a sequence having at least 60, 65, 70, 75, 80, 85 or 90% identity with the amino acid sequence as set forth in SEQ ID NO: 7, SEQ ID NO: 8, SEQ ID NO: 9, SEQ ID NO: 10 or SEQ ID NO: 11, respectively; or a function-conservative variant or derivative thereof.

18. The method of claim 8 wherein the TREM-1 inhibitor is a TREM-1 peptide comprising or consisting of an amino acid sequence as set forth in SEQ ID NO: 7, SEQ ID NO: 8, SEQ ID NO: 9, SEQ ID NO: 10 or SEQ ID NO: 11; or a sequence having at least 60, 65, 70, 75, 80, 85 or 90% identity with the amino acid sequence as set forth in SEQ ID NO: 7, SEQ ID NO: 8, SEQ ID NO: 9, SEQ ID NO: 10 or SEQ ID NO: 11, respectively; or a function-conservative variant or derivative thereof.

19. The method of claim 8 wherein the TREM-1 inhibitor is a TREM-1 peptide having an amino acid sequence as set forth in SEQ ID NO: 7, SEQ ID NO: 8, SEQ ID NO: 9, SEQ ID NO: 10 or SEQ ID NO: 11 or a sequence having at least 60, 65, 70, 75, 80, 85 or 90% identity with the amino acid sequence as set forth in SEQ ID NO: 7, SEQ ID NO: 8, SEQ ID NO: 9, SEQ ID NO: 10 or SEQ ID NO: 11, respectively; or a function-conservative variant or derivative thereof.

20. The method of claim 11 wherein the TLT-1 peptide consists of from 6 to 20 amino acids or from 6 to 17 amino acids.

21. The method of claim 16 wherein the TREM-1 peptide consists of from 6 to 20 amino acids or from 6 to 17 amino acids.


SEQUENCE LISTING (12 sequences)

SEQ ID NO: 1 - protein, 234 aa, Homo sapiens
MRKTRLWGLLWMLFVSELRAATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTKGFSGTPGSNENSTQNVYKIPPTTTKALCPLYTSPRTVTQAPPKSTADVSTPDSEINLTNVTDIIRVPVFNIVILLAGGFLSKSLVFSVLFAVTLRSFVP

SEQ ID NO: 2 - protein, 17 aa, synthetic construct
  REGION 1..17: note = LR17
LQEEDAGEYGCMVDGAR

SEQ ID NO: 3 - protein, 12 aa, synthetic construct
  REGION 1..12: note = LR12
LQEEDAGEYGCM

SEQ ID NO: 4 - protein, 6 aa, synthetic construct
  REGION 1..6: note = LR6-1
LQEEDA

SEQ ID NO: 5 - protein, 6 aa, synthetic construct
  REGION 1..6: note = LR6-2
EDAGEY

SEQ ID NO: 6 - protein, 6 aa, synthetic construct
  REGION 1..6: note = LR6-3
GEYGCM

SEQ ID NO: 7 - protein, 17 aa, synthetic construct
  REGION 1..17: note = LP17
LQVEDSGLYQCVIQHPP

SEQ ID NO: 8 - protein, 12 aa, synthetic construct
  REGION 1..12: note = LP12
LQVEDSGLYQCV

SEQ ID NO: 9 - protein, 6 aa, synthetic construct
  REGION 1..6: note = LP6-1
LQVEDS

SEQ ID NO: 10 - protein, 6 aa, synthetic construct
  REGION 1..6: note = LP6-2
EDSGLY

SEQ ID NO: 11 - protein, 6 aa, synthetic construct
  REGION 1..6: note = LP6-3
GLYQCV

SEQ ID NO: 12 - protein, 311 aa, Homo sapiens
MGLTLLLLLLLGLEGQGIVGSLPEVLQAPVGSSILVQCHYRLQDVKAQKVWCRFLPEGCQPLVSSAVDRRAPAGRRTFLTDLGGGLLQVEMVTLQEEDAGEYGCMVDGARGPQILHRVSLNILPPEEEEETHKIGSLAENAFSDPAGSANPLEPSQDEKSIPLIWGAVLLVGLLVAAVVLFAVMAKRKQGNRLGVCGRFLSSRVSGMNPSSVVHHVSDSGPAAELPLDVPHIRLDSPPSFDNTTYTSLPLDSPSGKPSLPAPSSLPPLPPKVLVCSKPVTYATVIFPGGNKGGGTSCGPAQNPPNNQTPSS